(19) United States
(12) Patent Application Publication
(10) Pub. No.: US 2024/0366320 A1
(43) Pub. Date: Nov. 7, 2024

(54) ROBOTIC SURGICAL TOOL ALIGNMENT

(71) Applicant: LEM Surgical AG, Bern (CH)

(72) Inventor: Yossi BAR, Muri bei Bern (CH)

(73) Assignee: LEM Surgical AG, Bern (CH)

(21) Appl. No.: 18/742,817

(22) Filed: Jun. 13, 2024

Related U.S. Application Data
(63) Continuation-in-part of application No. PCT/IB2022/058982, filed on Sep. 22, 2022.
(60) Provisional application No. 63/291,460, filed on Dec. 20, 2021.

(51) Int. Cl. A61B 34/30; A61B 34/20; A61B 90/00

(52) U.S. Cl. CPC A61B 34/30; A61B 34/20; A61B 90/361; A61B 2034/2059; A61B 2034/301; A61B 2034/305; A61B 2560/0437

(57) ABSTRACT

A robotic surgical system includes multiple robotic arms which hold, place and/or manipulate surgical access tools, such as ports, for placing and positioning other surgical tools, such as end effectors, cameras, and markers or other navigation elements, for use in performing a robotic surgical procedure. The robotic system will reposition the ports as the surgical procedure progresses to optimize performance of the robotic surgical procedure.

### CROSS-REFERENCE TO RELATE APPLICATIONS

This application is a continuation-in-part of PCT Application PCT/IB2022/058982 (Attorney Docket No. 67551-704.601), filed on Sep. 22, 2022, which claimed the benefit of U.S. Provisional Application 63/291,460 (Attorney Docket No. 67551-704.101), filed on Dec. 20, 2021, the full disclosures of each of which are incorporated herein by reference.

### BACKGROUND

Field. The disclosed technology relates generally to surgical robots and methods for their use in surgical procedures. More particularly, the disclosed technology relates to systems and methods for robotically aligning multiple surgical tools with a target site in a patient anatomy for performing a surgical procedure at the target site.

Robotic surgery is well known in the art with surgical robots operating in a variety of ways.

Many surgical robots, such as the da Vinci® robotic surgery system from Intuitive Surgical Systems, are “teleoperated,” where a user positions arms of the surgical robot and controls operation of surgical tools held by the arms in real time from a remote workstation while monitoring the procedure on a display. Other surgical robots function at least partially autonomously where a robotic controller positions the robotic arm and manipulates the surgical tools based on preoperative and intraoperative imaging of the patient anatomy with minimal or no real-time user input. For example, the surgical robot may control robotic functions based on predetermined control algorithms or rules while tracking the patient's anatomy and the surgical tools in a surgical space with cameras or other sensors. The surgical arms and tools can be kinematically controlled with a defined surgical robotic coordinate space.

Robotic surgery is used in a variety of surgeries, including but not limited to prostate and other urologic surgeries, cardiac bypass and other coronary procedures, gynecological surgeries, and the like. Of particular to the technologies described herein, multi-armed surgical robots have been developed for use in spinal and other orthopedic surgeries. For example, the Sapien™ surgical robot is being developed by LEM Surgical AG, the assignee of the present application, for spinal and other orthopedic surgeries. The Sapien™ surgical robot is a multi-armed robot mounted on a mobile cart configured to be placed beneath a surgical table to locate at least one tool-bearing arms on each side of the table. The basic structure and function of the Sapien™ surgical robot are described in PCT application no. PCT/IB2022/052297 (published as WO2022/195460), commonly owned with the present application, the full disclosure of which is incorporated herein by reference. The Sapien™ surgical robot is useful for performing a variety of procedures, particularly those which required two or more tools to operate within a common surgical space.

When operating two or more tools together within a common surgical space, it is essential that the location of each tool within the space be precisely known by the surgical robotic controller. While the general position of each tool relative to the patient anatomy can be separately monitored by the robot's cameras and other tracking sensors, the position of each tool relative to the other tool(s) will often be more difficult to track, particularly if the tools are initially misaligned by even a small amount. For example, if the initial tool positions differ from the kinematically determined positions, such errors will be propagated as the tools are kinematically repositioned during the procedure. As the tools will often be working in tandem, such positioning errors can be a significant challenge.

A second challenge arises during percutaneous or transcutaneous advancement of a surgical tool during a robotic surgical procedure. Advancement of a surgical tool through tissue can inadvertently damage nerves and related structures, particularly in spinal procedures with a high nerve density, for example when performing discectomy procedures.

For these reasons, it would be desirable to provide improved systems and methods for aligning two or more robotically controlled surgical tools at any time during a robotic surgical procedure, particularly at the outset before a procedure using the tool has begun. Such system and methods should assure that the relative positions of the two or more tools within a common surgical space is precisely known, thus allowing the tools to be kinematically moved relative to each other with precision. It would be further desirable if the systems and methods would further allow nerve monitoring when the surgical tools are being advanced though tissue. At least some of these objectives will be met by the technologies disclosed herein.

2. Background Art. PCT Publication Nos. WO2021/250580; WO2021/050767; and US2021/36135 describe surgical robots having multiple arms for deploying multiple tools;

Commonly owned publications and applications include PCT application no. PCT/IB2022/052297 (published as WO2022/195460); PCT application no. PCT/IB2022/058986 (published as WO2023/067415); PCT application no. PCT/IB2022/058972 (published as WO2023/118984); PCT application no. PCT/IB2022/058978 (published as WO2023/144602); PCT application no. PCT/IB2022/058980 (published as WO2023/152561); PCT application no. PCT/IB2022/055047 (published as WO2023/223215); PCT application no. PCT/IB2022/058988 (published as WO2023/237922; PCT application no. PCT/IB2023/055439; PCT application no. PCT/IB2023/056911; PCT application no. PCT/IB2023/055662; PCT application no. PCT/IB2023/055663; U.S. application No. 63/524,911; and U.S. application No. 63/532,753, the full disclosures of each of which are incorporated herein by reference.

### SUMMARY

In a first aspect, the disclosed technology provides a robotic surgical system comprising a chassis, first and second surgical robotic arms, and a central control unit. The first surgical robotic arm is disposed on the chassis and configured to hold a first surgical tool having a first axially oriented working channel. The second surgical robotic arm is disposed on the chassis and configured to hold a second surgical tool having a second axially oriented working channel. The central control unit is configured (a) to kinematically position the first and second robotic arms in a surgical robotic coordinate space and (b) to detect proximity of first and second positioning probes advanced through the first and second working channels when said probes are distally advanced from said the first and second working channels into a subcutaneous surgical site.

In some instances, at least one of the first and second probes comprises a proximity sensor at a tip thereof. For example, the central control unit is configured to detect a proximity of at least 2 mm, of at least 5 mm, of at least 10 mm, or at least 20 mm.

In some instances, the central control unit is configured to detect contact of first and second elongate probes. For example, the central control unit may be configured to detect contact of the first and second probes based upon one or more of electrical, mechanical, visual, and fluoroscopic assessment. More specifically, the central control unit may be configured to measure electrical resistance between the first and second probes, wherein a drop in the measured resistance indicates contact between the probes.

In some instances, the robotic surgical systems will further comprise the first and second probes that use being used for positioning. In other instances, the positioning probes could be provided separately from the other components of the robotic system, for example comprising commercially available stiff wires, rods, tubes, or other similar elongate structures.

Suitable stiff wires and other rod-like and/or tubular structures will typically have a diameter in a range from 1 mm to 5 mm and a length from 10 cm to 50 cm.

In some instances, the wires are electrically conductive.

In some instances, the disclosed systems further comprise a third robotic arm where, for example, the third surgical robot arm may be configured to hold an imaging device or sensor.

In some instances, the chassis comprises a mobile cart, where for example the mobile cart may be configured to be positioned beneath a surgical table so that the first and second surgical robotic arms are configured to lie on opposite sides of the surgical table.

In some instances, the mobile cart may include a single structure configured to be removably positioned beneath the surgical table.

Alternatively, the chassis comprises two or more mobile structures configured to be assembled beneath the surgical table.

In a second aspect, the disclosed technology provides a method for positioning first and second surgical robotic tools prior to performing a robotic surgical procedure in a surgical robotic coordinate space, where the method comprises positioning a first robotic arm of a surgical robot to locate a distal portion of a first positioning probe carried in a working channel of a first surgical tool in the surgical robotic coordinate space and positioning a second robotic arm of the surgical robot to locate a distal portion of a second positioning probe carried by a working channel of a second surgical tool in the surgical robotic coordinate space. A proximity between the distal portions of the first and second probes is determined, and the determined proximity is compared with a kinematically predicted proximity to confirm proper kinematic positioning of the surgical robotic arm and tool.

In some instances, determining proximity may comprise detecting proximity between said distal portions using at least one proximity detector on at least one of the first and second positioning probes.

In some instances, confirming that the distal portions of the first and second positioning probes are both present in the target site comprises detecting proximity between said distal portions, where for example “proximity” may comprise a minimum distance between said distal portions of 2 mm or less.

In some instances, confirming that the distal portions of the first and second positioning probes are both present in the target site comprises detecting contact between said distal portions, where detecting contact comprises assessing one or more of electrical, mechanical, visual, and fluoroscopic characteristics of the probes.

In a particular instance, detecting contact of the first and second probes comprises measuring electrical resistance between the first and second probes, wherein a drop in the measured resistance indicates contact between the probes is present.

In some instances, the first and second positioning probes comprise straight, stiff wires.

In some instances, the wires have a diameter in a range from 1 mm to 5 mm and a length from 10 cm to 50 cm.

In some instances, the methods further comprise removing the first and second positioning probes from the working channels of the first and second surgical tools and advancing one or more operative tools through at least one of the working channels and using the operative to perform a surgical procedure.

In some instances, the first and second surgical tools comprise tubular cannulas. Cannulas comprise tubular structures having a central lumen or “working channel” typically intended for advancement a working tool which is then manipulated manually or otherwise by the surgeon. In many instances, the cannulas of the disclosed technologies can be similar or identical to cannulas used in conventional endoscopic and laparoscopic procedures.

Typically, cannulas will be used for positioning “working” surgical tools configured to perform a procedure, such as drilling, milling, driving screws and other fasteners, radiofrequency or other ablations, and the like. In such cases, each cannula will be used to direct or align the working tool with a common target site Confirming the proper positions of the cannulas helps assure that the working surgical tools will be properly oriented when advanced to the target site.

In other instances, proper orientation of the cannulas will allow the cannulas themselves to be further advanced toward the surgical target site while minimizing the risk of damage of sensitive tissues, such as nerves. For example, the small diameter probes of the disclosed technologies can be advanced past the patient's nerve roots to assure proper cannula alignment before advancing the larger diameter cannulas when preforming disc resections or other spinal procedures.

In some instances, the first and second surgical tools comprise endoscopes having working channels.

In some instances, the first and second surgical tools comprise endoscopes and said endoscopes are used to perform a surgical procedure.

In still further aspects, the disclosed technology provides a mobile bi-lateral robotically controlled surgical system. Specifically, the disclosed system is a centrally coordinated and synchronized robotic system for spinal robotic endoscopy procedures, optionally for bilateral spinal robotic endoscopy procedures. The system comprises multiple robotic arms that each can hold, place and/or manipulate at least one end effector, port, camera/sensor or navigation/tracking element for use in a spinal endoscopy procedure. The ports may take the form of working channels or trocars for providing access to the surgical field for end effectors. The end effectors may include any surgical tools useful for performing spinal endoscopy procedures. The cameras/sensors and navigation/tracking elements (including, but not limited to, force/torque sensing, CT, MRI etc.) are for providing guidance for the movement of the robotic arms and deployment of the ports, end effectors and tools.

The disclosed technology comprises multiple robotic arms which access and visualize the surgical field in an automatic and safe way because they are robotically synchronized. In some embodiments, there may be three robotic arms, two of which place, guide and/or hold ports and one holding a navigation camera. In such an embodiment, the arms holding the ports may, after the ports have been placed, bring and manipulate other end effectors in the surgical field. In such an embodiment, the first arm may optionally position a port and then control the use of, for example, a drilling tool. The second arm may optionally position a second port and then control the use of, for example, a grasping tool. A third arm may optionally hold a camera that provides an image of the process from an optimal distance and angulation. The camera is able to operate from optimal distance and angulation because it is sized appropriately and its deployment on an appropriately sized and positioned robotic arm. Optionally, the robotic arms may also hold additional imaging or navigation cameras to provide redundancy and diversity of information. Also optionally, the robotic arms and/or the ports or end effectors may have active or passive markers placed on them that may assist the robotic system in positioning the robotic arms, the ports and/or the end effectors.

In some embodiments, the synchronized movement of the robotic arms is enabled by the interaction of the navigation cameras with active or passive markers that are placed at the beginning of the procedure on portions of the patient's anatomy. The movement of the robotic arms is synchronized by a central control unit from a single base that knows where the arms are based upon the navigation information provided by the various markers and the one or more cameras.

Accordingly, in various embodiments of the disclosed system, passive or active markers may be used to assist in navigation during a spinal endoscopy procedure that may employ a bilateral approach. These procedures may require the placement of multiple passive or active markers on the patient's anatomy or on the robotic arms, end effectors or ports. In particular embodiments, miniature markers may be preferred. Portions of the patient's anatomy may be relatively small and so to place multiple markers on different anatomical portions, it may be advantageous to use relatively small markers (1 cm or less in size). When using small markers, it may be advantageous to have the one or more cameras be deployed quite close to the surgical field, for example at a distance of 50 cm or less from the surgical field, and also at an advantageous angulation relative to the surgical field so that the marker(s) can be visualized. It may also be advantageous to place smaller markers on the robotic arms, end effectors or ports so that they do not obscure each other or aspects of the surgical field. This arrangement can then provide appropriate navigation information to the central control unit and provide for coordinated movement of the robotic arms in their placement and maintenance of the surgical ports and use of the end effectors. The disclosed approach allows for synchronized operation of the multiple robotic arms, and in many embodiments three arms may be chosen. One robotic arm may hold, position and maintain one endoscopic port. A second robotic arm may hold, position and maintain a second endoscopic port which may also include an endoscopic camera. A third robotic arm may position and hold a navigation camera. The robotic arms responsible for placing the endoscopic ports may also optionally deploy end effectors to the surgical field through the endoscopic ports.

In some embodiments, two robotic arms may simultaneously guide two ports in a bilateral approach to both sides of the patient and, thus, to both sides of the relevant surgical field. The bi-lateral ports may be conventional endoscopy ports of e.g. 5-10 mm in diameter. In an alternative related embodiment, three robotic arms may simultaneously guide three ports in a multilateral approach to multiple aspects of the surgical field. While this is theoretically enabled by the centralized control of the present robotic system, there may be limited applications in surgery. In alternative related embodiments, the at least two robotic arms may sequentially guide the at least two ports rather than simultaneously guiding them.

In various embodiments, an additional robotic arm mechanically connected to the same chassis of as the at least two robotic arms that guide the endoscopy ports may position and hold a navigation camera. The navigation camera may view the entire surgical field, and in particular the two ports that are held by the two other robotic arms in most bilateral spinal endoscopy procedures.

In some embodiments, guide wires that confirm accurate placement of the ports may be used. In one example, a 1-3 mm guide wire is used and can be inserted into a port after it has been placed or preferably before. In many spinal endoscopic procedures, the port/cannula is inserted just below the delicate nerve root with the intention of minimal contact with it. It will be advantageous to place a thin instrument/wire in said trajectory before inserting the wider port all the way and close to the nerve root and after proper position verification only than inserting the wider diameter port. The guide wire is significantly smaller in diameter than the port and, thus, carries much less risk to the patient. More over the guide wire is thin and rigid therefore inhibits less resistance with the soft tissue, meaning less deviations and more accurate placement. The guide wires can be used to confirm proper placement of the endoscopic ports with higher accuracy and less risky. Moreover, the guide wires may be equipped with electrophysiological sensor (passive or active) which can provide another layer of safety. Another verification method can be to insert the wire through the cannula from one side and from the other side to insert though he cannula an endoscopic camera. This camera can serve for visual verification of the successful arrival of the first wire or the current insertion and/or manipulation of a surgical tool through the first arm. The endoscopic camera can of course be inserted from both sides, together or separately.

In some embodiments, a method of using the robotic system of the disclosed technology is provided in the context of a robotic bilateral spinal endoscopy procedure. In this disclosed method, after the robotic arms carrying the ports have reached the appropriate position and trajectory as determined by pre-operative planning and navigation provided by a camera on a third robotic arm (with the possible assistance of miniature markers), the ports are inserted into the patient's soft tissue and insertion is stopped at a safe position with respect to the spinal anatomy (and in particular with respect to delicate nervous structures). Before advancing the ports any further, the surgeon may insert guide wires (e.g., 1-3 mm wide) into the ports and advance them further through the patient's soft tissue, eventually puncturing the disc annulus on either side of the surgical field. In one example of usage of this technique if the trajectory of insertion of both ports and guide wires has been planned and executed correctly, the guide wires will come into contact with each other in the designated anatomical field (i.e., disc space). The robotic system can determine that the guide wires have come into contact with each other by electrical conductivity for example. This will, in turn, allow the robotic system and with that also to the surgeon to determine that it Is safe to advance the ports further into the surgical field to the point where relevant tools or end effectors can be advanced through the ports to perform the surgical procedure.

In various embodiments, the guide wires can be deployed manually by the surgeon or completely automatically by the surgical robot. The disclosed embodiments take advantage of multiple feedback loops to ensure precision and safety in the performance of the bilateral robotic spinal endoscopy procedure. The movement of the robotic arms is robotically synchronized to the greatest possible level of precision because the arms are all co-mounted on a single chassis that has a central control unit. Robotic navigation is provided by one or more cameras that are deployed by one or more robotic arms that are also co-mounted on the same single chassis and are also controlled by the same central control unit. Thanks to the special system morphology, the distance between the robotic camera/sensor and the navigated/tracked markers is minimal. Due to the close vicinity of the camera/sensor, the tracked markers are extremely accurate since they are small and upholds minimal movement due to its size. Also, a closed loop of electrical conductivity allows the robot and/or the user to confirm appropriate placement of the endoscopy ports with minimal risk so that the surgeon knows that it is safe to proceed with the other phases of the spinal endoscopy procedure.

All of these needs and elements benefit tremendously from the central coordination and synchronized control of the single-cart, multi-arm, non-teleoperated robotic system. Based on the placement of appropriately sized markers and the placement of navigation cameras at an appropriate distance and orientation to the target anatomy and the markers, movement of the robotic arms carrying end effectors and cameras can be coordinated to provide for a safe and precise robotic spinal bi-lateral endoscopy procedure.

### BRIEF DESCRIPTION OF THE DRAWINGS

FIG. 1 shows a surgical robot having first and second surgical robotic arms which carry surgical access cannulas that can be aligned using the technology disclosed herein, in accordance with some embodiments.

FIG. 2 is a top view of a bilateral spinal procedure using the tool alignment system described herein, in accordance with some embodiments.

FIG. 3 is a side view of a bilateral discectomy procedure using the tool alignment system described herein, in accordance with some embodiments.

FIGS. 4A and 4B illustrate first and second embodiments of positioning probes in accordance with the disclosed technology, in accordance with some embodiments.

FIG. 5 is a perspective view of a pair of surgical robotic arms carrying cannulas for use in a bilateral discectomy procedure prior to alignment, in accordance with some embodiments.

FIG. 6 is a perspective view of the surgical robotic arms and cannulas of FIG. 5 with a positioning probe placed into each cannula, in accordance with some embodiments.

FIG. 7 shows the positioning probes of FIG. 6 being brought into contact proximate a target disc to confirm proper positioning prior to performing the bilateral discectomy procedure, in accordance with some embodiments.

FIG. 7A is a detailed view taken along line 7A-7A in FIG. 7, in accordance with some embodiments.

FIG. 8 shows the positioning probes of FIG. 6 being brought into proximity above a target disc to confirm proper positioning prior to performing the bilateral discectomy procedure, in accordance with some embodiments.

FIG. 8A is a detailed view taken along line 8A-8A in FIG. 8, in accordance with some embodiments.

### DETAILED DESCRIPTION

With reference now to the figures and several representative embodiments, the following detailed description is provided.

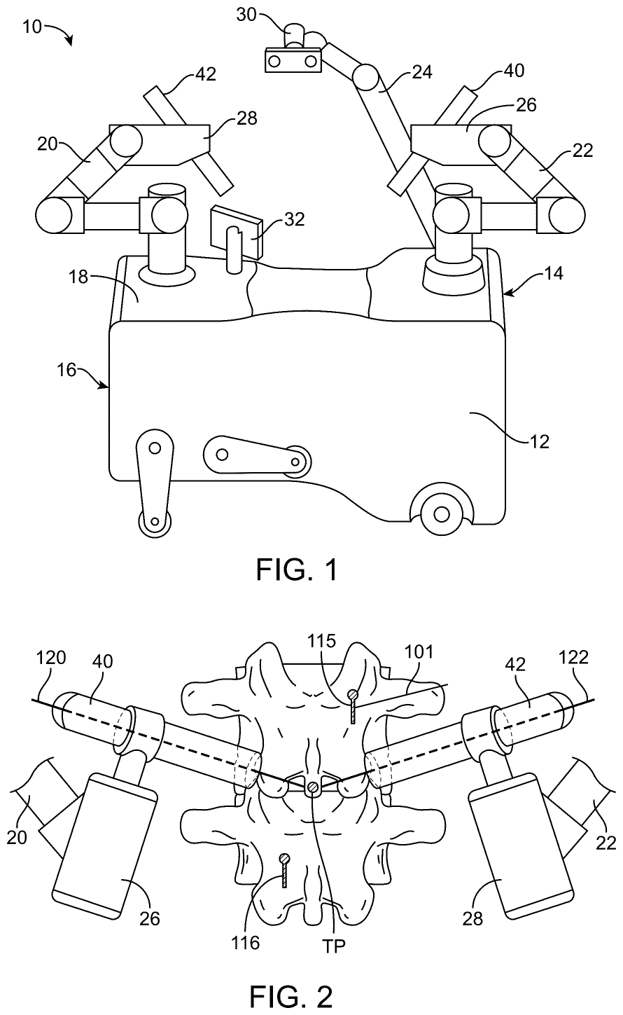

An exemplary robotic surgical system 10 intended particularly for use in the methods of the disclosed technology is shown in FIG. 1, in accordance with some embodiments. The robotic surgical system 10 comprises a chassis 12, typically a single, rigid frame which provides a base or platform for three robotic arms 20, 22 and 24 that are placed relatively far apart on opposite longitudinal ends 14 and 16 of an upper surface 18 of the chassis 12, typically approximately one meter apart, thus allowing for desirable attributes such as reachability, maneuverability, and an ability to apply significant force. In the illustrated embodiment, robotic surgical arms 20 and 22 are on the first end 14 of the chassis 12 and robotic surgical arm 22 is on the second end 16 of the chassis. The chassis is preferably but not necessarily mobile, e.g., being in the form of a mobile cart as described in commonly owned WO2022/195460, previously incorporated herein by reference. In other embodiments and implementations, the surgical arms 20, 22 and 24 could be mounted on a base or other structure of a surgical table. For performing tool alignment in accordance with the disclosed technology, the robotic surgical arms can be located on a stable platform that allows the arms to be moved kinematically or otherwise within a common robotic coordinate system under the control of a surgical robotic controller, typically an on-board controller have a user interface, such as display screen 32.

The single, rigid chassis of the disclosed technology can usually comprise a single mobile cart, as disclosed for example in commonly owned PCT application no. PCT/IB2022/052297 (published as WO2022/195460), the full disclosure of which has been previously incorporated herein by reference. In other instances, however, the single, rigid chassis may comprise separate modules, platforms, or components, that are assembled at or near the surgical table, as described for example in commonly owned PCT Application PCT/EP2024/052353, entitled Integrated Multi-Arm Mobile Surgical Robotic System, filed on Jan. 29, 2024, the full disclosure of which is incorporated herein by reference. The only requirement of the single, rigid chassis is that it provide a stable base for all the surgical arms so that they may be accurately and precisely kinematically positioned and tracked by the surgical robotic controller in a single surgical robotic coordinate space.

The chassis 12 of the robotic surgical system 10 can typically be configured to be temporarily placed under a surgical table (not shown) when performing the robotic surgical procedure, allowing the robotic surgical system 100 to be stored remotely before and after the procedure. The robotic arms 20, 22, and 24 may optionally be configured to be retracted into the chassis 101 of the robotic surgical system, allowing the system to be moved into or out of the surgical field in a compact configuration. The first robotic arm 20 can hold a first gripper 26 configured to hold an elongate tool having an axially aligned internal lumen or working channel, such as a cannula 40. The second robotic arm 22 can hold a second gripper 28 also configured to removably hold a cannula 42 or other elongate tool having an axially aligned lumen or working channel.

Referring now to FIG. 2, a bilateral spinal procedure is being performed on or near a portion of a patient's spinal bony anatomy including an upper vertebra portion 101 and a lower vertebra portion 102 near a disc space 103, which one of skill in the art will realize is directly adjacent to the delicate nerve roots.

The surgeon may optionally wish to place active or passive markers 115, 116 on the bony anatomy at or near in time to the beginning of the surgical procedure. The placement of these markers 115, 116 can assist a centrally coordinated robotic navigation system (not shown but described in commonly owned PCT application no. PCT/IB2021/055051 (published as WO2021/250580), the full disclosure of which has been previously incorporated herein by reference) to position robotic arms 20 and 22 which hold ports 40 and 42, typically tubular cannulas suitable for positioning surgical tools therethrough. The markers 115 and 116 may optionally be relatively small (1 cm or less in size) so that they don't interfere with the view of the surgical field obtained by the surgeon or the robotic navigation system.

The robotic navigation camera 30 (FIG. 1) is positioned to view the surgical field, including the markers 115, 116, and thus can assist in guiding the robotic arms 20 and 22 to the relevant portion of the surgical field. The robotic navigation camera 30 can be held within one meter or less of the patient's anatomy of interest without interfering with the surgeon's view and while appropriately visualizing the anatomy and the surgical markers.

The first port 40 and second port 42 are positioned and held by port holders, such as grippers 28 and 30, which are, in turn, held by robotic arms 20 and 22, respectively. The cannulas may comprise any tubular structure suitable for holding and aligning an “active” surgical tool intended to perform a surgical intervention, such as a drill, a cutter, a grinder, an electrosurgical tool, forceps, or the like. In some instances, the cannulas can comprise could be conventional 5 mm or 10 mm endoscopy ports.

The cannulas 40 and 42, or other ports, can be robotically positioned to “nominally” align their lumens or working channels with a “target point” TP, for example to a position just outside the disc space 103 in the surgical space. Typically, the robotic controller 32 will kinematically position the surgical arms 20 and 22 so that the longitudinal axes of the cannulas 40 and 42 (the centerlines of the associated lumen or working channel of each cannula) are directed with the target point TP.

In order to confirm that the cannulas 40 and 42 have been properly aligned by the surgical robot, first and second positioning probes 120 and 122, typically electrically conductive wires, may be advanced through the cannulas 40 and 42, respectively. If the probes intersect at the target point TP, proper alignment has been confirmed. If the probes do not intersect, the surgical robotic arms 20 and 22 can be repositioned, as described further below.

Suitable electrically conductive wires can typically have a width, usually a diameter, in a range from 1 mm to 3 mm and, when intended for percutaneous or transcutaneous use, can be configured to be advanced through tissue with minimum risk of nerve or other injury, e.g., have a rounded or tapered distal end. The robotic system may generate a signal if and when the probes 120 and 122 have intersected at the target point TP, e.g., energizing a signaling circuit when electrical contact is established between the electrically conductive wires.

Note that the inner diameter of the cannula 40 and/or 42 will often be much larger than the outer diameter of the probe 120 and/or 122, preventing accurate alignment of the probe with a centerline of the cannula. In such cases, the cannulas 40 and 42 may include a second, appropriately sized “alignment” lumen so that the probe will be properly constrained and oriented. Alternatively, a “centering” fixture can be provided for temporary placement in the cannula lumen to constrain and align the probe.

Once proper alignment has been established, the cannulas 40 and 42 can be advanced along the same path as the probes 120 and 122 and then used in a desired procedure. Alternatively, the cannulas may be used without advancement to introduce working tools for performing a procedure.

In all instances, initial positioning of the cannulas 40 and 42 may be adjusted to accommodate movement in that patient anatomy, typically by optical tracking of the markers 115 and 116 with cameral 30 (FIG. 1), as described, for example, in commonly owned PCT Publication WO2022/195460, the full disclosure of which has been previously incorporated herein by reference.

The use of electrically conductive probes has a further advantage as it allows “nerve monitoring” as the probes are being introduced, helping to avoid the delicate nerve roots in the adjacent anatomy, as described for example in US Publication No. 2007/0293782 and U.S. Pat. No. 7,920,922.

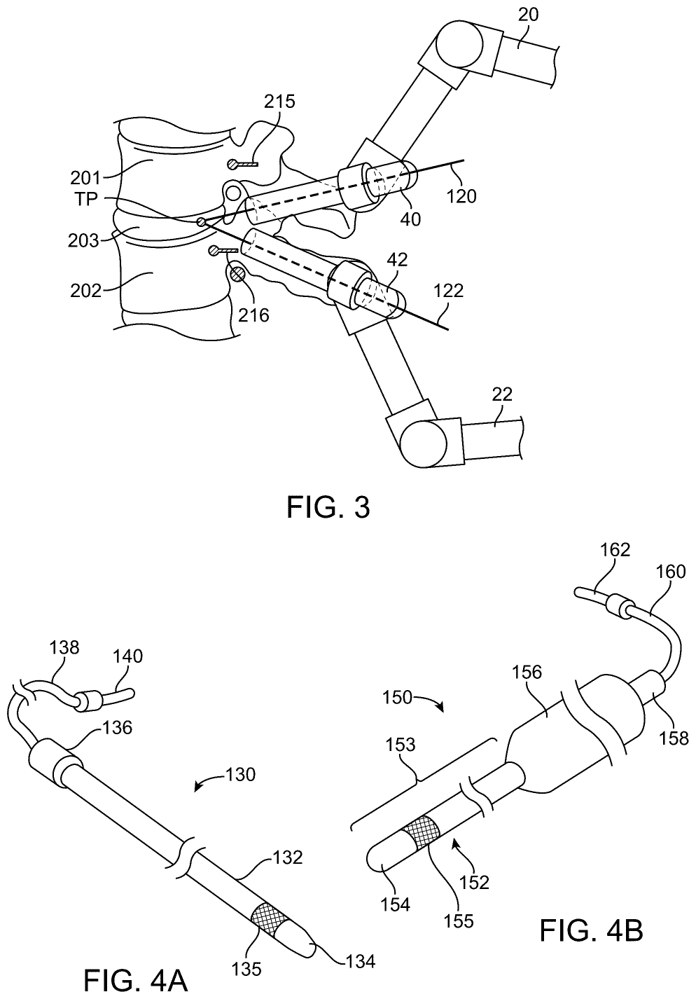

FIG. 3 is a side view of a discectomy procedure using the surgical robot 10 of FIG. 1, in accordance with some embodiments. The surgical procedure is being performed on or near a portion of a patient's spinal bony anatomy including an upper vertebra portion 201 and a lower vertebra portion 202. In particular, the surgical procedure is being performed in or near the disc 203, which one of skill in the art will realize is directly adjacent to the delicate nerve roots.

Active or passive markers 215, 216 are placed on the bony anatomy at or near in time to the beginning of the surgical procedure. The placement of these markers 215, 216 can assist the surgical robotic controller 32 to position robotic arms 20, 22, and 24 which hold instruments and cameras at or adjacent to the patient's target anatomy.

As shown in FIG. 3, the target point TP is in the disc space 203. The cannulas 120n and 122 can be directed at the disc space target point in the same manner as described with respect to FIG. 2, in accordance with some embodiments. After cannula positioning and alignment has been confirmed, appropriate disc resection tools can be introduced through each of the cannulas and used to resect and remove disc tissue. The robotic system can maintain alignment of the cannulas even as the patient anatomy moves.

Referring now to FIG. 4A, a first exemplary positioning probe 140 includes a straight shaft 132 having a uniform diameter, typically from 1 mm to 3 mm, along its length, typically from 10 cm to 50 cm. The shaft 132 has a blunt, tapered tip 134, e.g., similar to an obturator, to allow the shaft to be atraumatically advanced through soft tissue if needed. The shaft 132 typically comprises a metal or other electrically conductive material, although proximal portions of the shaft may be partially or fully covered by an electrical insulator. A distal region proximate the tip 134 can typically be free of insulation to allow electrical contact with tissue and/or a second positioning probe to establish electrical conductivity as described elsewhere herein. The positioning probe usually has a hub 136 at its proximal end, and a cable 138 and a plug connector 140 may be provided when the positioning probe is used to electrically detect a second positioning probe and/or is used for nerve monitoring.

Additionally, the positioning probe 130 may comprise a proximity detector 135 at or near the distal tip 134 thereof. Suitable proximity detectors will be able to sense the presence of nearby objects, such as a second positioning probe, without physical contact. For example, inductive and capacitive sensors will be able to detect the metal in other positioning probes. Proximity detectors using an inductive coil are preferred for incorporation into the positioning probes of the disclosed technology. Such proximity detectors will be able to detect and determine both the presence and the distance of the adjacent positioning probe.

Referring now to FIG. 4B, a second exemplary positioning probe 150 includes a straight shaft 152 having a non-uniform diameter with a small-diameter distal section 153 and a large-diameter proximal section 156. The shaft 152 has a blunt, tapered tip 154, e.g., similar to an obturator, to allow the shaft to be atraumatically advanced through soft tissue if needed. The small-diameter distal section 153 typically has a diameter from 1 mm to 3 mm, while the large diameter proximal section can have a diameter selected to fit closely within a lumen or working channel of the cannula or other tool being aliened, typically from 3 mm to 12 mm. The total length of the shaft 152 can typically be from 10 cm to 50 cm. The shaft 152 typically comprises a metal or other electrically conductive material, although proximal portions of the shaft may be partially or fully covered by an electrical insulator. A distal region proximate the tip 154 can typically be free of insulation to allow electrical contact with tissue and/or a second positioning probe to establish electrical conductivity as described elsewhere herein. The positioning probe 150 may also comprise a proximity sensor 155 and will usually have a hub 158 at its proximal end, and a cable 160 and a plug connector 162 may be provided when the positioning probe is used to electrically detect a second positioning probe and/or is used for nerve monitoring.

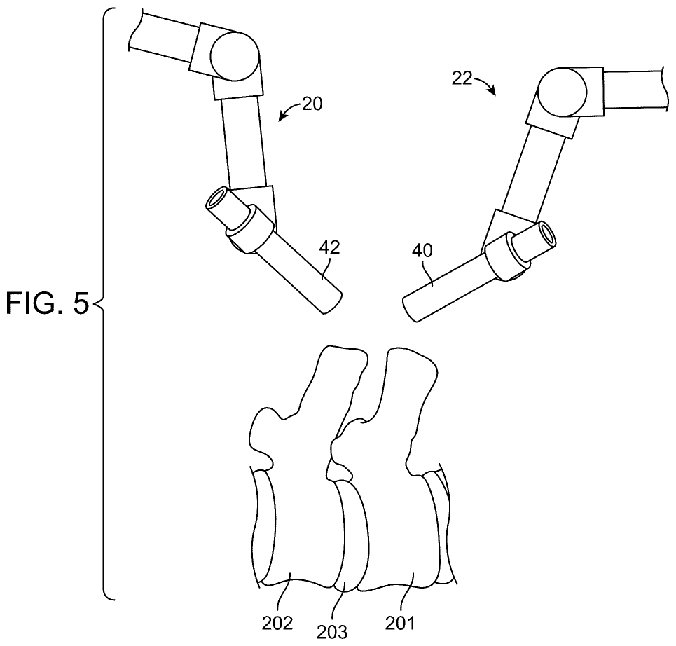

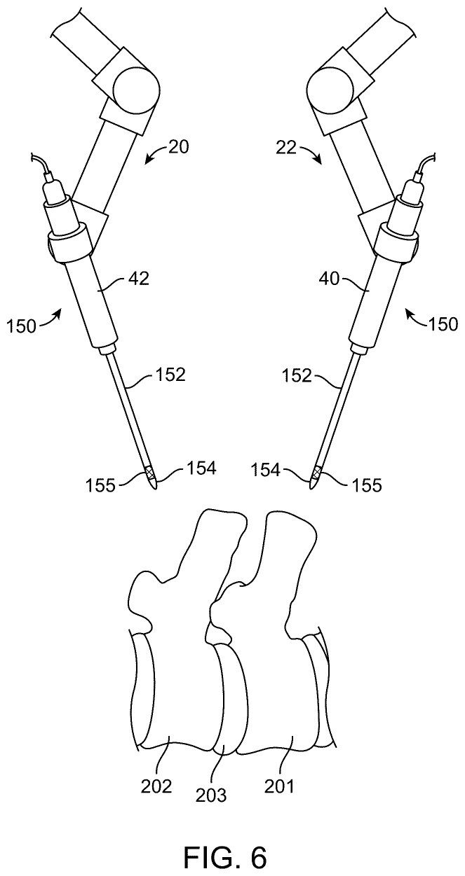

Referring now to FIG. 5, at the outset of a procedure, first and second robotic surgical arms 20 and 22 may hold the first cannula 40 and second the cannula 42 at random locations within the surgical robotic coordinate space and will have empty working channels, e.g., lumens. Before loading operative surgical tools, e.g., those tools which will be used in performing the robotic surgical procedure, positioning probes 150 will be placed into each cannula so that tips 154 extend distally away from the cannulas. As shown in FIG. 6, the position of each positioning probe 150 including the probe tip 154 can be kinematically determined by the robotic controller 32 (FIG. 1) in a conventional manner. The robotic controller 32 can kinematically track the positions of the robotic surgical arms 20 and 22 and can calculate the positions of the cannulas 40 and 42 within the surgical robotic coordinate space based on their known dimensions which can be provided to the controller. The kinematically determined positions will usually be highly accurate, but in some cases the arm and/or cannula positions may have fallen out registration which can lead to imprecision in the surgical procedure to follow.

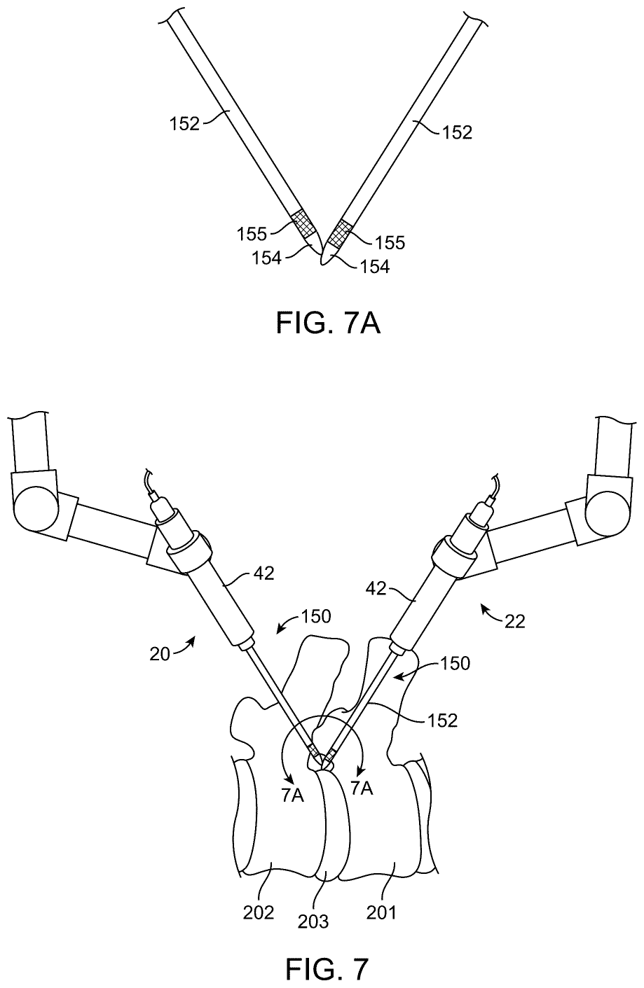

Positioning of the robotic surgical arms 20 and 22 and cannulas 40 and 42 can be checked and, if necessary, adjusted using positioning probes 130 and 150 employing either of two general approaches. As shown in FIGS. 7 and 7A, the probe tips 154 can be brought into contact at a kinematically predicted location in the surgical robotic coordinate space. While it is preferred that the location be at or near a surgical target site, shown as the patient's disk in FIG. 6, the location could theoretically be anywhere in the surgical robotic coordinate space. So long as the tips 154 of the probes 150 come into contact, which can be determined in any of the ways described previously, it can be assumed that all components of the surgical robot and surgical tools are properly kinematically registered and aligned, and the robotic surgical procedure can proceed. If the distal tips do not come into contact, the surgical arms and/or the cannula will need to be repositioned and the system rechecked.

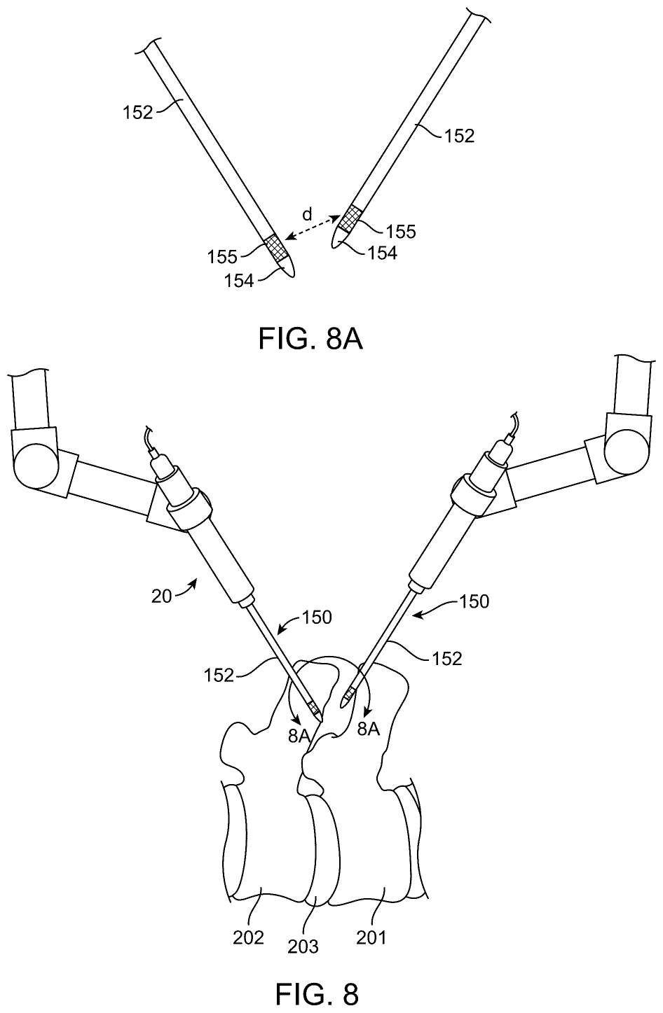

Proper kinematic positioning of the surgical robotic components can also be checked without bringing the probes 150 into contact. As shown in FIGS. 8 and 8A, the probe tips 154 can be brought into proximity, typically within typically within 20 mm, more typically within 10 mm, often within 5 mm, and preferably within 2 mm based as kinematic determined by the controller 32. Once positioned, either or both proximity sensors 155 can be used to determine an actual distance d between the distal portions of the probes. So long as the actual, measured distance d is the same as or within a threshold amount of the kinematically determined distance, proper alignment of the robotic arms and cannulas can be assumed. If the actual position falls outside this threshold expectation, the system may have to be recalibrated.

The disclosed embodiments take advantage of multiple feedback loops to ensure precision, efficiency and safety in the performance of the bilateral robotic spinal endoscopy procedure. The movement of the robotic arms is robotically synchronized to the greatest possible level of precision because the arms are all co-mounted on a single chassis that has a central control unit. Robotic navigation is provided by one or more cameras that are deployed by one or more robotic arms that are also co-mounted on the same single chassis and are also controlled by the same central control unit. Also, a closed loop of electrical conductivity allows the robot and/or the user to confirm appropriate placement of the endoscopy ports so that the surgeon knows that it is safe to proceed with the other phases of the spinal endoscopy procedure. All of these feedback loops also provide great efficiency in the performance of the robotic bilateral endoscopy procedure because when the surgeon is assured of safety and precision, they can also move through the procedure with confidence, and thus, relative speed.

In particular, the disclosed embodiment demonstrates the benefits of a bilateral approach to robotically coordinated spinal endoscopy. The disclosed system minimizes or removes altogether many of the risks associated with conventional spinal endoscopy. The system allows for much more precise placement of working channels, thus minimizing risk to nervous system structures. Also, the use of markers that are seen by the robotic system allows for the placement of trocars and movement of instruments with far less risk to the patient while still avoiding excessive radiation exposure to the patient.

All of these needs and elements benefit tremendously from the central coordination and synchronized control of the disclosed single-cart, multi-arm, non-teleoperated robotic system. Based on the placement of appropriately sized markers and the placement of navigation cameras at an appropriate distance and orientation to the target anatomy and the markers, movement of the robotic arms carrying end effectors and cameras can be coordinated to provide for a safe and precise robotic spinal bi-lateral endoscopy procedure. The centrally coordinated robotic navigation system provided by the Applicant as part of the disclosed system may be premised on the notion of mounting multiple robotic arms on a single, central chassis, wherein the central chassis also comprises a central control unit. The central control unit can coordinate the movements of three or more robotic arms that deploy surgical instruments and navigation cameras and are guided by navigation information provided by the navigation cameras and active or passive markers. In the disclosed technology, the central control unit can coordinate the movement of two robotic arms deploying port holders with additional instrumentation and/or endoscopic cameras in it and one or more robotic arm holding a navigation camera. The system is then able to guide surgical ports, held by the port holders, to spinal anatomy of interest through navigation information provided by the navigation camera and optionally placed active or passive markers.

List of Reference Numbers.

| | |
|---|---|
| 10 | Robotic surgical system |
| 12 | Chassis |
| 14 | First end |
| 16 | Second end |
| 18 | Upper surface |
| 20 | Robotic surgical arm (first) |
| 22 | Robotic surgical arm (second) |
| 24 | Robotic surgical arm (third) |
| 26 | First gripper |
| 28 | Second gripper |
| 30 | Navigation camera |
| 32 | Display/Controller |
| 40 | First cannula |
| 42 | Second cannula |
| 101 | Upper vertebra |
| 102 | Lower vertebra |
| 103 | Disc Space |
| 115, 116 | Markers |
| 120 | First positioning probe |
| 122 | Second positioning probe |
| 130 | Positioning probe (First embodiment) |
| 132 | Probe shaft |
| 134 | Blunt tapered tip |
| 135 | Proximity sensor |
| 136 | Hub |
| 138 | Cable |
| 140 | Plug connector |
| 150 | Positioning probe (Second embodiment) |
| 152 | Probe shaft |
| 153 | Small-diameter distal section |
| 154 | Blunt tapered tip |
| 155 | Proximity sensor |
| 156 | Large-diameter proximal section |
| 158 | Hub |
| 160 | Cable |
| 162 | Plug connector |
| 201 | Upper vertebra |
| 202 | Lower vertebra |
| 203 | Disc |
| 215, 216 | Markers |

One of skill in the art will realize that several variations on the disclosed embodiments are possible while staying within the bounds of the disclosed technology. Solely by way of example, different variations in the number of navigation cameras, robotic arms, markers and end effectors can be used without departing from the scope of the disclosed technology. As another example, markers of varying sizes can be used. As yet another example, numerous variations of surgical tools and surgical approaches to bilateral spinal endoscopy can be employed without departing from the scope of the technology described herein. The embodiments provided are representative in nature.

1. A robotic surgical system comprising:
a chassis;
a first surgical robotic arm disposed on the chassis and configured to hold a first surgical tool having a first working channel;
a second surgical robotic arm disposed on the chassis and configured to hold a second surgical tool having a second working channel;
a central control unit configured to (a) kinematically position the first and second robotic arms in a surgical robotic coordinate space and (b) detect proximity of first and second positioning probes advanced through the first and second working channels when distal portions of said probes are distally advanced from said the first and second working channels into a subcutaneous surgical site.

2. The system of claim 1, wherein the working channels are aligned on an axis of their respective surgical tools.

3. The system of claim 3, wherein the working channels and probes are straight.

4. The system of claim 1, wherein at least one of the first and second positioning probes comprises a proximity sensor at a tip thereof, wherein the central control unit detects proximity based on a signal from the proximity detector.

5. The system of claim 4, wherein the central control unit is configured to detect a proximity of at least 2 mm, of at least 5 mm, of at least 10 mm, or at least 20 mm.

6. The system of claim 1, wherein the central control unit is configured to detect contact of first and second elongate probes.

7. The system of claim 6, wherein detecting proximity comprises detecting contact of the first and second probes based upon one or more of electrical, mechanical, visual, and fluoroscopic assessment.

8. The system of claim 7, wherein the central control unit is configured to measure electrical resistance between the first and second probes, wherein a drop in the measured resistance indicates contact between the probes.

9. The system of claim 1, further comprising the first and second probes.

10. The system of claim 9, wherein the first and second probes comprise straight, stiff wires.

11. The system of claim 10, wherein the wires have a diameter in a range from 1 mm to 5 mm and a length from 10 cm to 50 cm.

12. The system of claim 11, wherein the wires are electrically conductive.

13. The system of claim 1, further comprising a third robotic arm.

14. The system of claim 13, wherein the third surgical robot arm is configured to hold an imaging device or sensor.

15. The system of claim 1, wherein the chassis comprises a mobile cart.

16. The system of claim 15, wherein the mobile cart is configured to be positioned beneath a surgical table so that the first and second surgical robotic arms are configured to lie on opposite sides of the surgical table.

17. The system of claim 15, wherein the mobile cart includes a single structure configured to be removably positioned beneath the surgical table.

18. The system of claim 15, wherein the chassis comprises two or more mobile structures configured to be assembled beneath the surgical table.

19. A method for positioning first and second surgical robotic tools prior to performing a robotic surgical procedure in a surgical robotic coordinate space, said method comprising:
positioning a first robotic arm of a surgical robot to locate a distal portion of a first positioning probe carried in a working channel of a first surgical tool in the surgical robotic coordinate space;
positioning a second robotic arm of the surgical robot to locate a distal portion of a second positioning probe carried by a working channel of a second surgical tool in the surgical robotic coordinate space;
determining a proximity of the distal portion of the first probe to the distal portion of the second probe; and
comparing the determined proximity with a kinematically predicted proximity to confirm proper kinematic positioning of the surgical robotic arm and tool.

20. The method of claim 19, wherein determining proximity comprises detecting proximity between said distal portions using at least one proximity detector on at least one of the first and second positioning probes.

21. The method of claim 20, wherein proximity comprises a distance between said distal portions of 2 mm or less.

22. The method of claim 19, wherein determining proximity comprises detecting contact between said distal portions.

23. The method of claim 22, wherein detecting contact of the first and second probes comprises assessing one or more of electrical, mechanical, visual, and fluoroscopic characteristics.

24. The method of claim 22, wherein detecting contact of the first and second probes comprises measuring electrical resistance between the first and second probes, wherein a drop in the measured resistance indicates contact between the probes is present.

25. The method of claim 19, further comprising removing the first and second positioning probes from the working channels of the first and second surgical tools and advancing one or more operative tools through at least one of the working channels and using the operative to perform a surgical procedure.

26. The method of claim 25, wherein the first and second surgical tools comprise tubular cannulas.

27. The method of claim 25, wherein the first and second surgical tools comprise endoscopes having working channels.

28. The method of claim 19, wherein the first and second surgical tools comprise cannulas and wherein the cannulas are advanced beneath a patient's nerve root in the patient's spine before performing a procedure on the spine with surgical tools introduced through the cannulas.

29.-43. (canceled)
